(19) United States
(12) Patent Application Publication
(10) Pub. No.: US 2002/0064500 A1
(43) Pub. Date: May 30, 2002

(54) Radiotracers for in vivo study of acetylcholinesterase and alzheimer's disease

(76) Inventors: J. James Frost, Baltimore, MD (US); Robert F. Dannals, Sparks, MD (US); John Musachio, Lutherville, MD (US); Ursula Scheffel, Baltimore, MD (US); Anabella Villalobos, Niantic, CT (US); Badreddine Bencherif, Towson, MD (US)

Correspondence Address: PENNIE AND EDMONDS, 1155 AVENUE OF THE AMERICAS, NEW YORK, NY, 100362711

(21) Appl. No.: 09/561,486

(22) Filed: Apr. 28, 2000

Related U.S. Application Data
(60) Provisional application No. 60/132,113, filed on Apr. 30, 1999.

(51) Int. Cl. A61M 36/14; A61K 51/00

(52) U.S. Cl. 424/1.69

(57) ABSTRACT

Methods for detecting acetylcholinesterase in a brain of a patient, comprising administering to the patient a detectable amount of a radiolabeled compound of a class of benzisoxazoles or a pharmaceutically acceptable salt thereof, are disclosed herein. The methods are useful for diagnosing, estimating the severity of, or monitoring the progression of a dementia, such as Alzheimer's disease, in a patient. In a preferred embodiment, the benzisoxazole is:

[chemical structure]

This application claims the benefit of U.S. Provisional Application No. 60/132,113, filed Apr. 30, 1999, the entire contents of which are incorporated herein by reference.

### BACKGROUND OF THE INVENTION

This invention relates to methods for detecting acetylcholinesterase in the brain of a patient and for diagnosing, estimating the severity of, and monitoring the progression of a dementia, such as Alzheimer's disease, in a patient.

Alzheimer's disease is the most common form of both senile and presenile dementia in the world and is recognized clinically as relentlessly progressive loss of memory and intellectual function and disturbances in speech (Merritt, 1979, A Textbook of Neurology, 6th edition, pp. 484-489 Lea & Febiger, Philadelphia). Alzheimer's disease begins with mildly inappropriate behavior, uncritical statements, irritability, a tendency towards grandiosity, euphoria, and deteriorating performance at work; it progresses through deterioration in operational judgement, loss of insight, depression, and loss of recent memory; and it ends in severe disorientation and confusion, apraxia of gait, generalized rigidity, and incontinence (Gilroy & Meyer, 1979, Medical Neurology, pp. 175-179 MacMillan Publishing Co.). Alzheimer's disease is found in about 10% of the population over the age of 65 and 47% of the population over the age of 85 (Evans et al., 1989, JAMA, 262:2551-2556).

The etiology of Alzheimer's disease is unknown. Evidence for a genetic contribution comes from several important observations such as the familial incidence, pedigree analysis, monozygotic and dizygotic twin studies, and the association of the disease with Down's syndrome (for review see Baraitser, 1990, The Genetics of Neurological Disorders, 2nd edition, pp. 85-88). Nevertheless, this evidence is far from definitive, and it is clear that other factors are involved.

The diagnosis of Alzheimer's disease at autopsy is definitive. Gross pathological changes are found in the brain, including low weight and generalized atrophy of both the gray and white matter of the cerebral cortex, particularly in the temporal and frontal lobes (Adams & Victor, 1977, Principles of Neurology, pp. 401-407 and Merritt, 1979, A Textbook of Neurology, 6th edition, Lea & Febiger, Philadelphia, pp. 484-489). The histological changes include neurofibrillary tangle (Kidd, 1963, Nature, 197:192-193; Kidd, 1964, Brain 87:307-320), which consists of a tangled mass of paired helical and straight filaments in the cytoplasm of affected neurons (Oyanagei, 1979, Adv. Neurol. Sci., 18:77-88 and Grundke-Iqbal et al., 1985, Acta Neuropathol., 66:52-61).

The diagnosis of Alzheimer's disease during life is more difficult than at autopsy since the diagnosis depends upon inexact clinical observations. In the early and middle stages of the disease, the diagnosis is based on clinical judgement of the attending physician. In the late stages, where the symptoms are more recognizable, clinical diagnosis is more straightforward. But, in any case, before an unequivocal diagnosis can be made, other diseases, with partially overlapping symptoms, must be ruled out. Usually a patient must be evaluated on a number of occasions to document the deterioration in intellectual ability and other signs and symptoms. The necessity for repeated evaluation is costly, generates anxiety, and can be frustrating to patients and their families. Furthermore, the development of an appropriate therapeutic strategy is hampered by the difficulties of rapid diagnosis, particularly in the early stages where early intervention could leave the patient with significant intellectual capacity and a reasonable quality of life. In brief, no unequivocal laboratory test specific for Alzheimer's disease has been reported.

Alzheimer's disease is associated with degeneration of cholinergic neurons, in the basal forebrain, which play a fundamental role in cognitive functions, including memory (Becker et al., 1988, Drug Development Research 12:163-195). Progressive, inexorable decline in cholinergic function and cholinergic markers in the brain of Alzheimer's-disease patients has been observed in numerous studies, and includes for example, a marked reduction in acetylcholine synthesis, choline acetyltransferase activity, acetylcholinesterase activity, and choline uptake. (Davis 1979, Brain Res. 171:319-327 and Hardy, et al., 1985, Neurochem. Int. 7:545-563). Even more, decreased cholinergic function may be an underlying cause of cognitive decline seen in Alzheimer's-disease patients (Kish et al., 1988, J. Neurol., Neurosurg., and Psych. 51:544-548). Choline acetyltransferase and acetylcholinesterase activities decrease significantly as plaque count rises, and, in demented subjects, the reduction in choline acetyl transferase activity was found to correlate with intellectual impairment (Perry, et al., Brit. Med. J. 25, November 1978, p. 1457).

A high-affinity, brain-selective acetylcholinesterase inhibitor suitable for radioimaging studies in humans has not been developed. Such a marker would be useful for diagnostic and prognostic aspects of Alzheimer's disease. Since reduced activity of acetylcholinesterase has been observed in the brain of patients with Alzheimer's disease, a decrease in acetylcholinesterase activity might indicate the progression of Alzheimer's disease. In this regard, several [¹¹C]-acetylcholinesterase inhibitors have been synthesized to selectively complex with acetylcholinesterase in the brain, whereafter the distribution of acetylcholinesterase can be determined by [¹¹C]-sensitive brain-imaging (e.g., imaging by position emission tomography (PET), Maziere 1995, Pharmac. Ther. 66:83-101). In one report, [¹¹C]-labeled tacrine ([¹¹C]-MTHA) was synthesized and administered to rodents and primates, but biodistribution imaging studies failed (Tavitian et al., 1993, Euro. J. Pharmacol. 236:229-238). In another example, the acetylcholinesterase inhibitor, [¹¹C]-physostigmine, was administered to rats and primates in an attempt to indicate acetylcholinesterase brain distribution in vivo via PET (Tavitian et al., 1993, Neuro. Report 4:535-538 and Planas et al., 1994, Neuroimage 1:173-180). But since brain-acetylcholinesterase quantification and binding kinetics are not available, it is difficult to predict what effect the short half life of physostigmine will have on its suitability as a PET imaging agent.

The benzisoxazole, below, is an example of a new class of acetylcholinesterase inhibitors that are highly potent and selective (Villalobos et al., Poster Presentation at the Annual Society of Neuroscience meeting, 1994).

[chemical structure]

This benzisoxazole has high affinity (IC₅₀ of 0.48 nM) and unprecedented selectivity (9300:1 brain acetylcholinesterase relative to butyrylcholinesterase, which is found primarily in red blood cells) for brain acetylcholinesterase. Although preliminary rodent biodistribution studies with the [¹¹C]-labeled version of the above benzisoxazole are encouraging, no PET imaging data of a complex of the above benzisoxazole and acetylcholinesterase in the human brain, has been published (Musacher et al., 1996, J. Nuclear Med. 37:5, Supplement, Abstract No. 155).

In summary, a need exists for a method to detect acetylcholinesterase in the brain of a patient. Moreover there exists a need to diagnose, monitor the progression of, and establish the severity of Alzheimer's disease. Although some efforts have focused on monitoring acetylcholinesterase activity, no acetylcholinesterase markers have proved effective for in vivo determination of acetylcholinesterase activity in the human brain.

### SUMMARY OF THE INVENTION

In one embodiment, the invention relates to a method for detecting acetylcholinesterase in a brain of a patient, comprising:

(a) administering to the patient a detectable amount of a compound of a general formula I

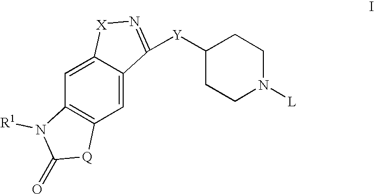

or a pharmaceutically acceptable salt thereof, the compound comprising one or more radioisotopic atoms selected from the group consisting of carbon-11, fluorine-18, iodine-123, and bromine-76, wherein:

Q is —(CH₂)ₘ—, —CH═CH—, —CHCH₃, —C(CH₃)₂, oxygen, sulfur, or —NR²;

X is oxygen or sulfur;

Y is —(CH₂)ₙ—;

L is phenyl or —(C₁-C₆)alkyl-phenyl, wherein said phenyl is optionally substituted with one or more —(C₁-C₆)alkyl or halo groups;

R¹ is —(C₁-C₆)alkyl;

R² is hydrogen or —(C₁-C₆)alkyl; and

n and m are independent integers ranging from 1 to 3;

with a proviso that the compound is not that of formula II

[chemical structure]

(b) imaging the brain to generate a brain image showing a distribution and relative amounts of acetylcholinesterase in the brain.

In another embodiment, the invention relates to a method for diagnosing, estimating the severity of, or monitoring the progression of a dementia in a patient, comprising:

(a) administering to the patient a detectable amount of a compound of a general formula I

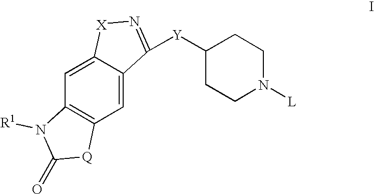

or a pharmaceutically acceptable salt thereof, the compound comprising one or more radioisotopic atoms selected from the group consisting of carbon-11, fluorine-18, iodine-123, and bromine-76, wherein:

Q is —(CH₂)ₘ—, —CH═CH—, —CHCH₃, —C(CH₃)₂, oxygen, sulfur, or —NR²;

X is oxygen or sulfur;

Y is —(CH₂)ₙ—;

L is phenyl or —(C₁-C₆)alkyl-phenyl, wherein said phenyl is optionally substituted with one or more —(C₁-C₆)alkyl or halo groups;

R¹ is —(C₁-C₆)alkyl;

R² is hydrogen or —(C₁-C₆)alkyl; and

n and m are independent integers ranging from 1 to 3;

with a proviso that the compound is not that of formula II

[chemical structure]

(b) imaging the brain of the patient to generate a brain image showing a distribution and relative amounts of acetylcholinesterase in the brain.

In a third embodiment, the invention relates to a method for detecting acetylcholinesterase in a brain of a patient, comprising:

(a) administering to the patient a detectable amount of a compound of a formula II

[chemical structure]

or a pharmaceutically acceptable salt thereof; and

(b) imaging the brain to generate a brain image showing a distribution and relative amounts of acetylcholinesterase in the brain.

In still another embodiment, the invention relates to a method for diagnosing, estimating the severity of, or monitoring the progression of a dementia in a patient, comprising:

(a) administering to the patient a detectable amount of a compound of a formula II

[chemical structure]

or a pharmaceutically acceptable salt thereof; and

(b) imaging a brain of the patient to generate a brain image showing a distribution and relative amounts of acetylcholinesterase in the brain.

The present invention may be understood more fully by reference to the figures, detailed description, and examples, which are intended to exemplify non-limiting embodiments of the invention.

### BRIEF DESCRIPTION OF THE DRAWINGS

FIG. 1 shows the images of trans-axial brain slices of a human patient, obtained by PET scanning as described in Example 3. The images show the relative concentration of a complex of acetylcholinesterase and compound II, where the color intensity correlates to the ratio of nCi/ccBRAIN/nCi/ccPLASMA (i.e., nanocurries per cubic centimeter of brain tissue divided by nanocurries per cubic centimeter of blood) according to the color scale to the right of the Figure.

FIG. 2 depicts the plot obtained in Example 4 showing the percentage of the administered dose of compound II/gram of brain tissue that is found in a particular brain region of male Charles River Mice post intravenous injection of the mice with 350 μCi of compound II versus time in minutes. The brain regions are abbreviated as follows: Str—striatum; Thal—thalamus; Rest—the rest of the brain; Ctx—parietal cortex; Cb—cerebellum; Hipp—hippocampus.

FIG. 3 depicts the plot obtained in Example 4 showing the difference between the values of the percentage of the administered dose of compound II/gram of brain tissue in a particular brain region and the value in the cerebellum versus time in minutes, post intravenous injection of Male Charles River Mice with 350 μCi of compound II. The brain regions are abbreviated as in FIG. 1.

FIG. 4 depicts the plot obtained in Example 5 showing the percentage of the administered dose of compound II/gram of brain tissue that is found in a particular brain region of Charles River Mice post intravenous injection of the mice with increasing doses of compound III followed by intravenous injection of the dose of compound II versus the dose in mg/kg of compound III. The brain regions are abbreviated as in FIG. 1.

### DETAILED DESCRIPTION OF THE INVENTION

The methods of the invention are useful for detecting acetylcholinesterase in human patients. Loss of acetylcholinesterase in humans is associated with brain disorders, such as dementia and epilepsy; muscle disorders; and disorders of the digestive system. The methods of the invention are particularly useful for detecting acetylcholinesterase in the brain of a patient suspected of suffering from a dementia, such as Alzheimer's disease, thereby allowing the diagnosis, estimating the severity of, and monitoring the progression of the dementia. Certain brain disorders and dementia, including Alzheimer's disease, are known to be accompanied by a decrease in acetylcholinesterase concentration in the brain. Thus, monitoring the concentration of acetylcholinesterase in the brain of a patient suspected of suffering from a brain disorder or dementia may allow diagnosis of the disorder or dementia, monitoring its progression, and/or estimating its severity.

The methods of the invention can be used to provide a brain image that shows the distribution and relative concentrations of acetylcholinesterase in a patient's brain, thereby allowing diagnosis, estimating the severity of, and analysis of the progression of a disorder or dementia in a patient. The methods of the invention can be used to diagnosis, estimate the severity, and monitor the progression of any dementia, known or to be discovered, that is accompanied by a detectable change in acetylcholinesterase concentration in the brain.

When administered to a patient, for clinical use, a compound of general formula I, compound II, or a pharmaceutically acceptable salt thereof, is preferably administered in isolated form. As used herein, “isolated” means that a compound of general formula I, compound II, or a pharmaceutically acceptable salt thereof, is separated from other components such as a synthetic organic chemical reaction mixture. Preferably, the compounds of general formula I, compound II, and a pharmaceutically acceptable salts thereof, are purified by conventional techniques. As used herein, “purified” means that when isolated, the isolate contains at least 95%, preferably at least 98%, of a single compound by weight of the isolate.

In one embodiment, the invention relates to a method for detecting acetylcholinesterase in a brain of a patient, comprising:

(a) administering to the patient a detectable amount of a compound of a general formula I

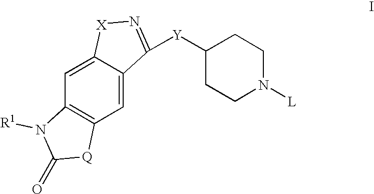

or a pharmaceutically acceptable salt thereof, the compound comprising one or more radioisotopic atoms selected from the group consisting of carbon-11, fluorine-18, iodine-123, and bromine-76, wherein:

Q is —(CH₂)ₘ—, —CH═CH—, —CHCH₃, —C(CH₃)₂, oxygen, sulfur, or —NR²;

X is oxygen or sulfur;

Y is —(CH₂)ₙ—;

L is phenyl or —(C₁-C₆)alkyl-phenyl, wherein said phenyl is optionally substituted with one or more —(C₁-C₆)alkyl or halo groups;

R¹ is —(C₁-C₆)alkyl;

R² is hydrogen or —(C₁-C₆)alkyl; and

n and m are independent integers ranging from 1 to 3;

with a proviso that the compound is not that of formula II

[chemical structure]

(b) imaging the brain to generate a brain image showing a distribution and relative amounts of acetylcholinesterase in the brain.

In another embodiment, the invention relates to a method for diagnosing, estimating the severity of, or monitoring the progression of a dementia in a patient, comprising:

(a) administering to the patient a detectable amount of a compound of a general formula I

[chemical structure]

or a pharmaceutically acceptable salt thereof, the compound comprising one or more radioisotopic atoms selected from the group consisting of carbon-11, fluorine-18, iodine-123, and bromine-76, wherein:

Q is —(CH₂)ₘ—, —CH═CH—, —CHCH₃, —C(CH₃)₂, oxygen, sulfur, or —NR²;

X is oxygen or sulfur;

Y is —(CH₂)ₙ—;

L is phenyl or —(C₁-C₆)alkyl-phenyl, wherein said phenyl is optionally substituted with one or more —(C₁-C₆)alkyl or halo groups;

R¹ is —(C₁-C₆)alkyl;

R² is hydrogen or —(C₁-C₆)alkyl; and

n and m are independent integers ranging from 1 to 3;

with a proviso that the compound is not that of formula II

[chemical structure]

(b) imaging the brain of the patient to generate a brain image showing a distribution and relative amounts of acetylcholinesterase in the brain.

In a third embodiment, the invention relates to a method for detecting acetylcholinesterase in a brain of a patient, comprising:

(a) administering to the patient a detectable amount of a compound of a formula II

[chemical structure]

or a pharmaceutically acceptable salt thereof; and

(b) imaging the brain to generate a brain image showing a distribution and relative amounts of acetylcholinesterase in the brain.

In still another embodiment, the invention relates to a method for diagnosing, estimating the severity of, or monitoring the progression of a dementia in a patient, comprising:

(a) administering to the patient a detectable amount of a compound of a formula II

[chemical structure]

or a pharmaceutically acceptable salt thereof; and

(b) imaging a brain of the patient to generate a brain image showing a distribution and relative amounts of acetylcholinesterase in the brain.

Preferred compounds of general formula I and pharmaceutically acceptably salts thereof, are those wherein R¹ is [¹¹C] methyl.

A second preferred group of compounds of general formula I and pharmaceutically acceptably salts thereof, are those wherein Y is —(CH₂)₂— and L is —CH₂—phenyl.

A still further preferred group of compounds of general formula I and pharmaceutically acceptably salts thereof, are those wherein X is —O—, Q is —CH₂—, and L is —CH₂—phenyl.

Another preferred group of compounds of general formula I and pharmaceutically acceptably salts thereof, are those wherein Q is —CH₂—, Y is —(CH₂)₂—, and L is —CH₂—phenyl.

In another preferred group of compounds of general formula I and pharmaceutically acceptably salts thereof, L is —CH₂—phenyl, in which the phenyl group is substituted with a halogen selected from the group consisting of I, F, Fluorine-18 [¹⁸F], and iodine-123 [¹²³I].

A particularly preferred compound useful for detecting acetylcholinesterase in the brain of a patient is 5,7-dihydro-7-[¹¹C]-methyl-3-[2-[1-(phenylmethyl)4-piperidinyl]ethyl]-6H-pyrrolo[3,2-f]-1,2-benzisoxazole-6-one, hereinafter compound II:

[chemical structure]

or a pharmaceutically acceptable salt thereof.

As used herein, the term “alkyl group” means a saturated, monovalent unbranched or branched hydrocarbon chain. Examples of alkyl groups include, but are not limited to, (C₁-C₆)alkyl groups. Examples of (C₁-C₆)alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl.

The term “phenyl” means —C₆H₅.

As used herein, “halogen” means fluorine, chlorine, bromine, or iodine. Correspondingly, the meaning of the term “halo” encompass fluoro, chloro, bromo, and iodo.

As used herein, the term “dose” means the quantity of a compound of general formula I or the quantity of compound II, or a pharmaceutically acceptable salt thereof, administered to the patient.

As used herein, the term “radioactivity” means the total radioactive activity, measured in millicurries, of a dose of a compound of general formula I, compound II, or a pharmaceutically acceptable salt thereof. The total radioactive activity of the dose is measured by methods well known in the art, for example using a dose calorimeter.

As used herein the term “patient” means a mammal, preferably a primate, more preferably a human, and most preferably a human suspected of suffering from a dementia or a human predisposed to a dementia. Optimally, the patient is a human suspected of suffering from Alzheimer's disease or a human predisposed to Alzheimer's disease.

The phrase “pharmaceutically acceptable salt,” as used herein includes, but is not limited to, salts of the basic amino group(s) present in compounds of general formula I and compound II. A compound of general formula I and compound II are basic and are thus capable of forming a wide variety of salts with various inorganic and organic acids. The acids that may be used to prepare pharmaceutically acceptable acid addition salts of such basic compounds are those that form non-toxic acid-addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A compound of general formula I and compound II may also form pharmaceutically acceptable salts with various amino acids, in addition to the acids mentioned above.

The compounds of general formula I, compound II, and pharmaceutically acceptable salts thereof, can be prepared by methods well known in the art. Exemplary procedures are disclosed in EP 976404; WO 9947131; WO 9925363; WO 9613505;WO 9304063; WO 9217475; U.S. Pat. Nos. 5,750,542; 5,538,984; and Villalobos et al., 1995, J. Med. Chem. 38:2802-2808, all of which citations are incorporated herein by reference. Those skilled in the art will recognize that synthetic procedures taught in the above references for the synthesis of compounds of general formula I, compound II, and pharmaceutically acceptable salts thereof, can be adapted to produce the corresponding radiolabeled compounds by introducing one or more radioactive atoms at appropriate steps in the synthesis. Starting materials useful for preparing the compounds of general formula I, compound II, and pharmaceutically acceptable salts thereof, and intermediates therefor, are commercially available or can be prepared by well known synthetic methods.

Scheme 1, below, illustrates a synthesis of compound II from 5,7-dihydro-3-[2-[1-(phenylmethyl)-4-piperidinyl]ethyl]-6H-pyrrolo[3,2-f]-1,2-benzisoxazole-6-one, hereinafter compound III.

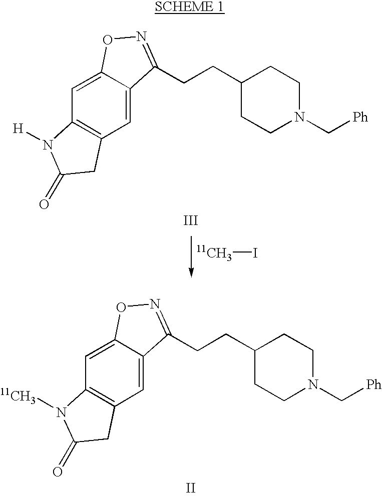

Compound III can be prepared as disclosed in WO 9217475 pp. 57-60, incorporated herein be reference. [¹¹C]—CH₃I can be prepared according to the procedure described in Musachio et al, 1996, J. Nucl. Med. 37:41 P. incorporated by reference herein. High specific radioactivity [¹¹C]-compound II can be synthesized by treatment of compound III with [¹¹C]-methyl iodide. Preferably, the reaction proceeds in the presence of tetrabutylammonium hydroxide (TBAH) and DMF. The reaction is advantageously run at a temperature of about 80° C. for a time of about 5 minutes. Yields range form about 10% to about 30%, typically from about 14% to about 24%.

According to the methods of the present invention, after administration to a patient, a compound of general formula I, compound II, or a pharmaceutically acceptable salt thereof, crosses the blood-brain barrier and enters the brain. The compound of general formula I, compound II, or a pharmaceutically acceptable salt thereof, forms a complex with acetylcholinesterase in the brain. Because the compounds of formula I, compound II, and pharmaceutically acceptable salts thereof, are radioactive, the complex can be imaged, thereby showing the presence, absence, distribution, or relative concentration of acetylcholinesterase in the brain. Any brain-imaging method, known or to be discovered, that is sensitive to the radioisotopes carbon-11 [¹¹C], fluorine-18 [¹⁸F], bromine-76 [⁷⁶Br], and iodine-123 [¹²³l], can be used to acquired a brain image showing the presence, absence, distribution, or the relative amounts of the complex. Examples of such imaging techniques include planar imaging, positron emission tomography (PET), and single photon emission computerized tomography (SPECT). Planar imaging, PET, and SPECT are well known to those of the art (e.g., see Frost J J, Mayberg H S: The Brain: Epilepsy. Principles of Nuclear Medicine, Second Edition, H N Wagner and Z Szabo, Eds. W. B. Saunders Company, pp 564-575, 1995; Maziere, 1995, Pharmac. Ther. 66:83; and Kilbourne, et al., 1996, Synapse 22:123, all three of which are incorporated herein by reference. Planar imaging is accomplished using a single flat camera that provides a 2-dimensional image of the radiolabel, while PET and SPECT provide 3-dimensional images. Using positron (β+) or γ-cameras, PET and SPECT can monitor the time course of regional tissue radioactivity, after administration of a compound labeled with a β+ (e.g., ¹¹C) or γ-photon-emitting radionuclide, respectively. PET and SPECT methodologies allow the performance of in vivo sequential studies, and radioactivity versus time can be plotted in selected brain regions of interest. These two methods are safe, non-invasive, and due to the short half-life of the radioisotopes used, weakly irradiating. The preferred brain imaging methods are PET and SPECT, more preferably PET. For PET studies, the main positron-emitting radionuclides useful for the labeling of acetylcholinesterase inhibitors are: carbon-11 [¹¹C], with a 20.4 min half-life; fluorine-18 [¹⁸F], with a 110 min half-life; and bromine-76 [⁷⁶Br], with a 16 hour half-life. All of these radionuclides should be prepared with very high specific radioactivity in a cyclotron. For SPECT studies, iodine-123 [¹²³I] is preferable to image the complex. The half-life of iodine-123 is 13.2 hr. This radioisotope is commercially available with very high specific radioactivity.

Absolute radiotracer quantitation in tissue is possible using routine PET and SPECT studies. Facilities capable of performing PET and SPECT imaging exist worldwide, for example, Northern California PET Imaging Center, Sacramento, Calif. and Yale-VA Positron Imaging Laboratory, West Haven, Conn. A list of these facilities is published by ICP, Institute for Clinical PET.

Preferably, imaging is commenced at the time of administration. Preferably, about 1 to about 35 scans are obtained with the PET or SPECT device within about 1 minute to about 4 days after administration, more preferably about 20 to about 30 scans within about 1 hour to to about 3 hours. As the dosage or sensitivity of the imaging device increases, the number of scans and scanning time can be reduced. But compounds labeled with radioisotopes with relatively long half lives, such as ¹⁸F or ¹²³l, can be imaged up to about 6 hours and 24 hours respectively after administration.

The compounds of general formula I, compound II, and pharmaceutically acceptable salts thereof, can be administered in the form of a pharmaceutical composition. In this case, the pharmaceutical composition should be administered to the patient as soon as possible after its preparation, preferably within 10 minutes, more preferably within 3 minutes. A further delay can result in a reduction of the compound's specific radioactivity and thus provide a less-informative brain image.

A patient suspected of having a dementia, such as Alzheimer's disease, will generally display symptoms well known to physicians. Genetic and other high-risk factors, such as family incidence of the disease can be taken into account by the physician.

Methods of administration of compounds of general formula I, compound II, pharmaceutically acceptable salts thereof, and pharmaceutical compositions thereof include, but are not limited to, intradermal, intramuscular, intraperitoneal, intravenous, subcutaneous, epidural, oral, sublingual, intranasal, intracerebral, intravaginal, transdermal, rectally, by inhalation, or topically, particularly to the ears, nose, eyes, or skin. Preferably, the mode of administration is intravenous injection, injection into arteries leading to the brain, or injection into the cerebral spinal fluid, more preferably, intravenous injection. The preferred cite of intravenous injection is the antecubital vein, but any accessible superficial vein is acceptable.

The pharmaceutical compositions can comprise a pharmaceutically acceptable vehicle. A pharmaceutically acceptable vehicle can take the form of a sterile solution, suspension, emulsion, tablets, pill, pellet, capsule, powder, or any other form suitable for administration. Examples of suitable pharmaceutical vehicles are described in Remington's Pharmaceutical Sciences 18th Edition, ed. Alfonso Gennaro, Mack Publishing Co. Easton, Pa, 1990. In a preferred embodiment, the pharmaceutical compositions are adapted for intravenous administration to human beings. Typically, pharmaceutical compositions for intravenous administration comprise sterile solutions containing an isotonic aqueous buffer. Where necessary, the compositions may also include a solubilizing agent. The preferred pharmaceutically acceptable vehicle for intravenous injection comprises U.S.P. injectable physiological (0.9% NaCl) saline solution and 8.4% U.S.P. injectable sodium bicarbonate, in a ratio of about 70% saline to 30% sodium bicarbonate solution volume to volume, and ammonium formate in an amount of about 50 mg/ml of vehicle. Preferably the pH of the vehicle is about 7.5. Suitable pharmaceutical vehicles can also include excipients such as glycerol, propylene glycol, starch, glucose, lactose, sucrose, gelatin, malt, rice, flour, chalk, silica gel, sodium stearate, glycerol monostearate, and talc. The pharmaceutical compositions, if desired, can also contain minor amounts of wetting or emulsifying agents, or pH buffering agents. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical compositions are administered by injection, an ampule of sterile water or saline can be provided and the additional ingredients added prior to injection.

The detectable amount of a compound of general formula I, compound II, or a pharmaceutically acceptable salt thereof, will be the dose capable of providing a brain image. The dose will depend on the sensitivity of the imaging device and the dose's radioactivity. Every imaging device has limitations in count rate and sensitivity. For example, if the dose is too high, the detector saturates and the resulting brain image is less useful. Thus, as the sensitivity of the imaging device increases, for example, with advances in technology, the dose of a compound of general formula I, compound II, or a pharmaceutically acceptable salt thereof, required for a useful brain image will decrease accordingly. The dose will also depend on the route of administration; the physical characteristics of the patient, such as height and weight; and the extent of the dementia and should be decided according to the judgment of the practitioner and each patient's circumstances. Preferably, the dose will have a radioactivity ranging from about 0.1 millicurrie to about 100 millicurries, more preferably, about 5 to about 50 millicurries, even more preferably, about 10 to about 30 millicurries, and most preferably, about 15 to about 25 millicurries.

Preferably, the dose will have low toxicity. In this regard, it is preferred that the amount of a compound of general formula I, compound II, or a pharmaceutically acceptable salt thereof, in the dose is as low as possible to provide a brain image. Toxicity can be measured using well-known toxicity models or subsequently during brain-imaging studies on human subjects. Preferably, the amount of a compound of general formula I, compound II, or a pharmaceutically acceptable salt thereof, in the dose will range from about 0.001 to about 1 micrograms per kilogram body weight of the patient, more preferably, from about 0.005 to about 0.5 micrograms per kilogram body weight, and most preferably, from about 0.01 to about 0.06 micrograms per kilogram body weight.

In a pharmaceutical composition comprising a pharmaceutically acceptable vehicle and a compound of general formula I, compound II, or a pharmaceutically acceptable salt thereof, the concentration of the compound in the pharmaceutical composition will generally range from about 1 μg/ml of pharmaceutically acceptable vehicle to about 15 μg/ml of pharmaceutically acceptable vehicle, more preferably, from about 2 μg/ml to about 8 μg/ml, most preferably, from about 5 μg/ml to about 7 μg/ml.

The following Examples are illustrative of the present invention. It is to be understood that the present invention is not limited to the specific details of the Examples provided below.

### EXAMPLE 1

### Synthesis of 5,7-Dihydro-3-[2-[1-(phenylmethyl)-4-piperidinyl]ethyl]-6H-pyrrolo-[3,2-f]-1,2-benzisoxazol-6-one maleate (i.e., the maleate salt of compound III).

[chemical structure]

### a) 5-Acetyl-1,3-dihydro-6-hydroxy-2H-indol-2-one

Acetyl chloride (4.09 ml, 0.0575 mol) was added to a slurry of aluminum trichloride (AlCl₃) (35.36 g, 0.265 mol) in carbon disulfide (CS₂) (250 ml). After 2-3 min, 6-methoxyoxindole (7.22 g, 0.0442 mol) was added. The resulting mixture was heated to reflux for 2.5 hours. Excess solvent was decanted and ice water was added carefully to the residue. The resulting mixture was stirred overnight. The pale yellow solid obtained was collected, washed with water and dried under high vacuum to give the above-titled compound (7.32 g, 87%). ¹H-NMR (DMSO-d₆) δ 13.0 (s, 1H), 10.8 (s, 1H), 7.70 (s, 1H), 6.30 (s, 1H), 3.40 (s, 2H), 2.54 (s, 3H).

### b) 5-Acetyl-1,3-dihydro-6-hydroxy-2H-indol-2-one, 5-oxime

An aqueous solution of hydroxylamine hydrochloride (8.26 g, 0.119 mol) and sodium acetate trihydrate (16.9 g, 0.124 mol) was added to a mixture of 5-acetyl-1 ,3-dihydro-6-hydroxy-2H-indol-2-one, formed in step a (9.88 g, 0.0517 mol) and EtOH (600 ml). The resulting mixture was refluxed for 20 hours. The hot reaction mixture was filtered and the solid collected was rinsed with ethanol. After drying, the title compound (10.11 g, 95%) was obtained as a pale yellow solid. ¹H-NMR (DMSO-d₆) δ 12.0 (s, 1H), 11.4 (s, 1H), 10.5 (s, 1H), 7.29 (s, 1H), 6.35 (s, 1H), 3.38 (s, 2H), 2.20 (s, 3H).

### c) 5-Acetyl-1,3-dihydro-6-hydroxy-2H-indol-2-one, 5-oxime acetate

A heterogeneous mixture of 5-acetyl-1,3-dihydro-6-hydroxy-2H-indol-2-one, 5-oxime formed in step b (7.15 g, 34.7 mmol) and acetic anhydride (55 ml) was heated at 80° C. for 2 hours. The cooled reaction mixture was filtered and the solid collected was rinsed with water. After drying, the above-titled compound (4.67 g, 54%) was obtained as a pale yellow solid. ¹H-NMR (DMSO-d₆) δ 11.3 (s, 1H), 10.6 (s, 1H), 7.35 (s, 1H), 6.44 (s, 1H), 3.41 s, 2H), 2.37 (s, 3H), 2,21 (s, 3H).

### d) 5,7-Dihydro-3-methyl-6H-pyrrolo[3,2-f]-1,2-benzisoxazol-6-one

A mixture of 5-acetyl-1,3-dihydro-6-hydroxy-2H-indol-2-one, 5-oxime acetate, formed in step c (4.48 g, 18.0 mmol), pyridine (14.6 ml, 180 mmol), and dimethylformamide (DMF) (660 ml) was heated at 125-130° C. for 4 hours. The cooled reaction mixture was poured over water and extracted with EtOAc (4 times). The combined organic layer was washed with water and brine and dried (MgSO₄), filtered, and concentrated. Purification by chromatography (50% EtOAc/hexanes→100% EtOAc) gave the above-titled compound (2.20 g, 65% yield) as a pale yellow-orange solid. M.p. (EtOAc): 264-265° C. (dec.); ¹H-NMR (DMSO-d₆) δ 10.8 (s, 1H), 7.60 (s, 1H), 6.98 (s, 1H), 3.57 (s, 2H), 2.47 (s, 3H).

### e) 4-[2-[5,7-Dihydro-6H-pyrrolo[3,2-f]-1,2-benzisoxazol-6-one-3-yl ]ethyl]-1-piperidinecarboxylic acid, 1-(1,1-dimethylethyl)ester

Freshly prepared 1M Lithium diisopropyl amide (LDA) in tetrahydrofuran (THF) (40.9 ml, 40.9 mmol) was quickly added dropwise to a cold (−78° C.) solution of 5,7-dihydro-3-methyl-6H-pyrrolo[3,2-f]-1,2-benzisoxazol-6-one formed in step d (2.33 g, 12.4 mmol) in THF (400 ml). Immediately after addition was complete, a solution of 4-iodomethyl-1-piperidinecarboxylic acid,1-(1,1-dimethylethyl) ester (4.42 g, 13.6 mmol) in dry THF (100 ml) was added in one portion. The resulting mixture was stirred at −78° C. for 4 hours. Saturated aqueous ammonium chloride (NH₄Cl) was added and the mixture was extracted with ethyl acetate (EtOAc) (3 times). The combined organic layer was washed with brine, dried over magnesium sulfate (MgSO₄), filtered and concentrated. Purification by chromatography (20%→30% EtOAc/CH₂Cl₂) gave recovered starting material (0.210 g, 9%) and the above-titled compound (2.75 g, 58%) as an off-white solid. ¹H-NMR (CDCl₃) δ 8.48 (s, 1H), 7.44 (s, 1H), 7.03 (s, 1H), 4.08-4.14 (m, 2H), 3.63 (s, 2H), 2,97 (t, 2H, J=7.8 Hz), 2.69 (brt, 2H, J=12.8 Hz), 1.74-1.84 (m, 4H), 1.46-1.55 (in, 1H), 1.46 (s, 9H), 1.18 (ddd, 2H, J=24.4 Hz), J=12.1 Hz, J=4.3 Hz).

### f) Synthesis of the maleate salt of Compound III.

Trifluoroacetic acid (TFA) (3.3 ml) was added dropwise to a cold (0° C.) solution of 4-[2-[5,7-dihydro-6H-pyrrolo[3,2f]-1,2-benzisoxazol-6-one-3-yl] ethyl]-1-piperidinecarboxylic acid, 1-(1,1-dimethylethyl) ester, formed in step e (0.50 g, 1.30 mmol) in CH₂Cl₂ (13 ml). After 30 min, the mixture was concentrated and excess TFA was removed by concentrating from toluene (2 to 3 times). The crude residue was dissolved in DMF (12.5 ml) and sodium carbonate (Na₂CO₃) (0.689 g, 6.50 mmol) and benzyl bromide (0.186 ml, 1.56 mmol) were added. The resulting mixture was stirred at room temperature for 4 hours. The reaction was filtered and the filtrate was concentrated in vacuo. The residue was dissolved in methylene chloride, washed with brine, and dried (MgSO₄), filtered, and concentrated. Purification by chromatography (CH₂Cl₂→10% methanol/CH₂Cl₂) gave the free-base form of the above-titled compound (i.e., compound III) (0.343 g, 70%) as a white solid. The corresponding maleate salt was prepared by adding a solution of maleic acid (0.061 g, 0.528 mmol) in ethanol (EtOH) (1 ml) to a solution of the free base (0.180 g, 0.48 mmol) in CH₂Cl₂ (10 ml). After concentrating, the salt was purified by recrystallization from isopropanol to give an off-white solid. Yield: 0.173 g, 73%; M.p. 194-195° C.; ¹H-NMR (DMSO-d₆) δ 10.82 (s, 1H), 7.65 (s, 1H), 7.48 (s, 5H), 7.00 (s, 1H), 6.03 (s, 1H), 4.24 (br s, 2H), 3.58 (s, 2H), 3.25-3.38 (m, 2H), 2.94 (t, 2H, J=7.6), 2.81-2.97 (m, 2H), 1.86-1.96 (m, 2H), 1.62-1.76 (m, 2H), 1.30-1.60 (m, 3H); calc'd for C₂₃H₂₅N₃O₂•C₄H₄O₄: C, 65.97; H, 5.95; N, 8.55. Found: C, 65.98; H, 6.04; N, 8.54.

### EXAMPLE 2

### Radiosynthesis, Purification, and Formulation of Compound II

A similar procedure has been described in Musachio et al, 1996, J. Nucl. Med. 37:41 P. incorporated by reference herein. The maleate salt of compound III, as prepared in Example 1f (2 mg), was dissolved in water (0.5 ml) to which was added 2 pasteur-pipet drops of 2N NaOH. The aqueous layer was extracted with diethyl ether (2×1 ml) and the extracts were passed through a Na₂SO₄ column (0.5 mm i.d.×2.5 cm). The ether filtrate was evaporated under a gentle stream of argon. The compound III, thus produced, in the form of a white film was redissolved in 200 μl of dimethylformamide (DMF) and transferred to a 1 ml septum sealed vial. The vial was cooled (−78° C.) and [¹¹C]-methyl iodide was passed into the reaction vessel by a stream of nitrogen carrier gas as follows:

Two liters of ultra high purity nitrogen (Matheson Gas Products) were bombarded with protons accelerated by a small biomedical cyclotron (Scanditronix RNP-16). [¹¹C]-carbon dioxide was formed by the reaction ¹⁴N(p,α)¹¹C. The target chamber of the cyclotron was connected to the chemical reaction vessel by ⅛″ stainless steel tubing. The apparatus for generating [¹¹C]-carbon dioxide consists of the following: (1) a conical glass vessel (length 50 mm, i.d.=5 mm) connected to a reaction vessel equipped with a water-cooled reflux condenser (length=50 mm, i.d.=50 mm) via Teflon tubing (i.d. 1.5 mm) and electrovalves (General Valve Corp, Series 2) interfaced to a small computer (Hewlett Packard HP-85) for valve sequencing; (2) a second conical vessel of similar dimensions for trapping [¹¹C]-methyl iodide; (3) two heat guns (150° C.); (4) a remote cooling (−78° C.) bath; (5) a high performance liquid chromatograph (Rheodyne Model 7126 injector, Waters Associates 6000 A pump, Waters Associates 6 μm, C-18 Nova-Pak, 30 cm×7.8 mm i.d. column) equipped with a ultra-violet detector (Waters Associates Model 440, 254 nM) and a flow radioactivity detector; and (6) a rotary evaporator modified for remote addition and removal of solutions. Upstream from this apparatus, there was a coil of stainless steel tubing (i.d.=2.2 mm) cooled by liquid nitrogen to retain [¹¹C]—CO₂ removed from the target under reduced pressure created by an oilless pump. Nitrogen was used as a sweep gas at a flow rate of 50 ml/min to sweep the [¹¹C]—CO₂ through the above apparatus. This apparatus was evacuated and purged with argon prior to each synthesis to minimize carrier carbon contamination. [¹¹C]—CO₂ produced by a 16 MeV proton irradiation of a nitrogen gas target was trapped in the cooled stainless steel coil following bombardment. The cooling bath was removed and the trapped CO₂ was bubbled into the conical vessel containing 3.0 mg lithium aluminum hydride in 600 μl of anhydrous tetrahydrofuran. After the level of radioactivity in the vessel reached a maximum, the vessel was heated with a heat gun to evaporate the tetrahydrofuran. Hydriodic acid (500 μL, 57% in water) was then added to the hot vessel. [¹¹C]-methyl iodide, thus produced, was transferred from the production apparatus by a stream of nitrogen carrier gas into a cooled solution (−78° C.) of about 1.0 mg of compound III, as prepared above in 200 μL anhydrous dimethylformamide. When the level of radioactivity reached a plateau, the stream of gas was stopped. Aqueous tetrabutylammonium hydroxide (5 μl, 0.4 M) was added to the reaction mixture via Hamilton microsyringe. The reaction mixture was heated in an 80° C. water bath for 5 minutes prior to quenching by addition of 0.2 ml of HPLC solvent consisting of 30:70 acetonitrile:0.1 M aqueous ammonium formate. The resulting mixture was injected onto a Waters Nova-Pak 18 6μ(7.8 mm×30 cm) semi-preparative column and eluted at a rate of 7 ml/min. The effluent from the column was monitored with a UV detector (254 nm, Waters module 440) and an in-line radioactivity detector (Ortec 449 ratemeter, 575 amplifier, 550 single channel analyzer, with a NaI (Tl) crystal). The fraction containing compound II and corresponding to the radioactive peak (t_{R}=5.2 min, k′=3.3) was collected in a rotary evaporator, and the acetonitrile and water were removed by evaporating to dryness under reduced pressure. The resulting residue was dissolved in sterile, normal saline (7 ml, 0.9% sodium chloride, injectable, U.S.P.); filtered through a sterile, 0.22 μM filter (Gelman Acrodisc, disposable filter assembly, sterile, nonpyrogenic) into a sterile, pyrogen free bottle (20 cc EVACUATED VIAL-sterile, pyrogen free; Medi-Physics/AmerSham Company, Arlington Heights, Ill. 60004); and diluted with sterile, sodium bicarbonate (3 ml, 8.4% sodium bicarbonate injectable, U.S.P.). The 10 ml dose thus produced was ready for injection into a patient. Such a composition comprises about 8 μl/ml of compound II.

The radiochemical yield of compound II was about 22% based on starting [¹¹C]-methyliodide (non-decay corrected, n=4). The specific radioactivity was about 1130 mCi/umol. Time of synthesis including composition and specific radioactivity determination was approximately 25 minutes. Compound II was of high radiochemical purity (>95%) and was sterile and pyrogen-free.

### EXAMPLE 3

### Imaging of Acetylcholinesterase in a Human Brain

In this study a dose of a composition comprising compound II was administered to a subject, and the subject's brain was imaged to determine the distribution and relative concentration of a complex of compound II and acetylcholinesterase. After allowing compound II to be discharged from the subject, a dose of a composition comprising donezepil hydrochloride in tablet form (ARICEPT, available commercially, for example from Pfizer)—a reversible inhibitor of acetylcholinesterase—together with a dose of a composition comprising compound II (as prepared in Example 2), was administered to the subject. The same imaging study was then performed.

The resulting distribution and relative concentration of the compound II/acetylcholinesterase complex with and without the reversible inhibitor, ARICEPT, were compared.

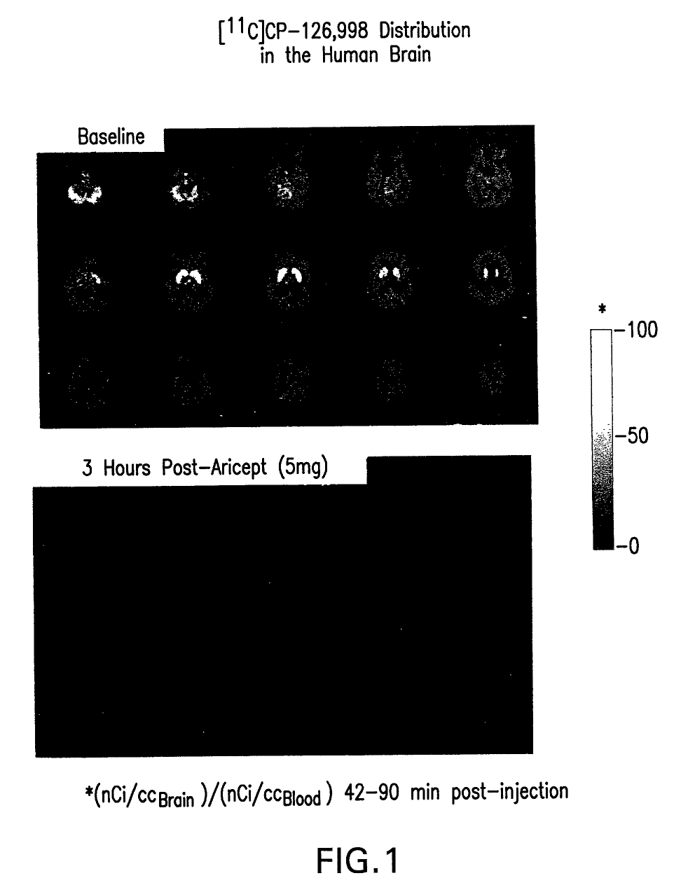

A healthy 30-year-old-male subject, about 5 feet 10 inches in height and 160 pounds in weight, was positioned in an a General Electric 4096+ PET scanner and 2-3 ml of the composition comprising compound II, as prepared in Example 2, was administered intravenously to his antecubital vein. A thermoplastic mask was used for PET positioning. Use of a thermoplastic mask is routine for PET studies to help immobilize the head and to provide spacial facial landmarks. To produce a brain image, PETwas begun, and 25 scans were obtained in 90 minutes. After each scan, heated venous blood samples were withdrawn from the back of the patient's hand, to measure the amount of the radiolabeled compound in the blood, in units of nCi/cc blood. The brain images were used to calculate nCi/ccBRAIN for each scan. The average of the ratio (nCi/ccBRAIN/nCi/ccPLASMA)_{control} (i.e., tissue radioactivity/plasma radioactivity or nanocurries per cubic centimeter of brain tissue divided by nanocurries per cubic centimeter of blood), over the scans collected after 42 minutes, for each area of the brain, are shown in Table 1. Only the scans collected after 42 minutes were used because after this time the ratio nCi/ccBRAIN/nCi/ccPLASMA showed the greatest difference among brain regions known to have different concentrations of acetylcholinesterase. The upper half of FIG. 1 shows the images of 15 trans-axial brain slices, obtained during the PET scanning. The images show the relative concentration of a complex of acetylcholinesterase and compound II according to the color intensity. The color intensity correlates to the ratio of nCi/ccBRAIN/nCi/ccPLASMA according to the color scale to the right of the Figure.

After 1-2 hours, to allow compound II to be discharged from the subject, a commercial tablet comprising 5 mg ARICEPT was administered to the subject orally. After 3 hours, the subject was positioned in an a General Electric 4096+ PET scanner. About 2 ml to about 3 ml of the composition comprising compound II, prepared in Example 2, was administered intravenously to the patient's antecubital vein. Brain images and brain time radioactivity curves were obtained in the same manner as above and the average nCi/ccBRAIN/nCi/ccPLASMA ratio was calculated for each area the brain. The data shown in Table 1 below is expressed as normalized uptake (tissue radioactivity/plasma radioactivity) post 5 mg ARICEPT. The lower half of FIG. 1 shows 15 trans-axial brain slice images, obtained during the PET scanning. Since at least a portion of the brain acetylcholinesterase was blocked by the ARICEPT, less acetylcholinesterase was available to complex with compound II. Hence, the images are much less intense than those obtained in the absence of ARICEPT.

TABLE 1
Uptake and Displacement of compound II in
the Brain of a Healthy Volunteer Subject

| | | Normalized | |
|---|---|---|---|
| | Normalized | uptake post | |
| | uptake | 5 mg ARICEPT | Percent |
| | (nCi/ccBRAIN/ | (nCi/ccBRAIN/ | displacement by |
| | nCi/ccPLASMA) | nCi/ccPLASMA) | ARICEPT 5 mg |
| Putamen | 61 | 26 | 57.4 |
| Caudate | 54 | 19 | 64.8 |
| Cerebellum | 47 | 13 | 72.3 |
| Medulla | 40 | N/A | N/A |
| Oblongata | | | |
| Pons | 36 | N/A | N/A |
| Thalamus | 33 | N/A | N/A |
| Hippocampus | 30 | N/A | N/A |
| Frontal cortex | 26 | 7 | 73.1 |
| Temporal | 26 | N/A | N/A |
| cortex | | | |
| Parietal cortex | 27 | 7 | 74.1 |
| Occipital | 23 | N/A | N/A |
| cortex | | | |

This study shows a 52% to 72% reduction in the ratio nCi/ccBRAIN/nCi/ccPLASMA when ARICEPT is used to bind acetylcholinesterase prior to administration of compound II versus administration of compound II alone. Thus this study confirms that compound II binds to acetylcholinesterase in a patient's brain to form a complex comprising compound II and acetylcholinesterase, and that the complex can be imaged by PET, showing the distribution and the relative concentration of acetylcholinesterase in the brain. No measurement was obtained for entries labeled “N/A”.

### EXAMPLE 4

### Kinetic Experiment

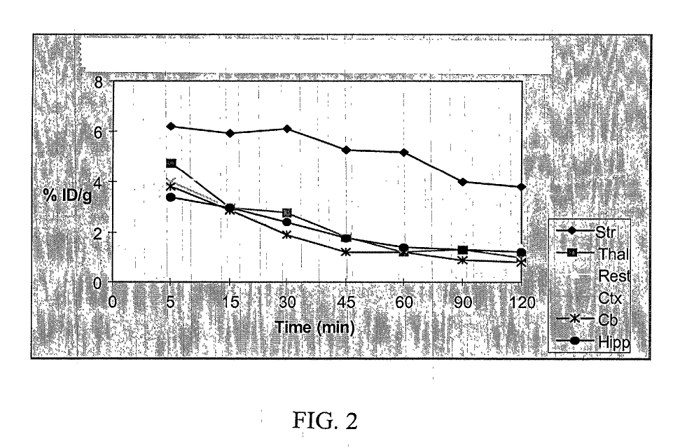

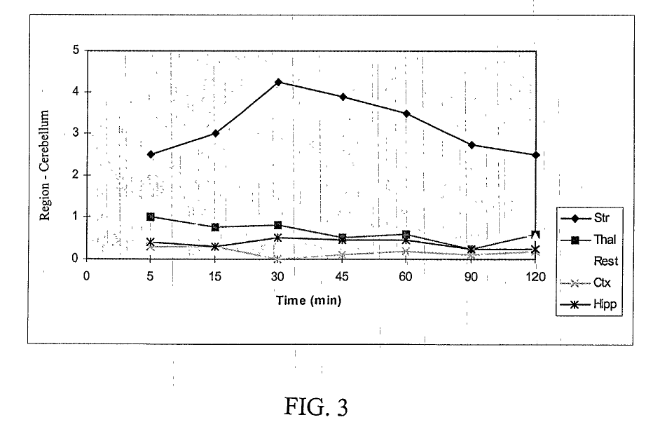

21 male Charles River mice (CD-1) were divided into 7 groups of 3 mice each. Each mouse was injected via a tail vein with approximately 350 μCi of compound II (10 μg). Each mouse was sacrificed by cervical dislocation at the following times post injection: group 1 at 5 minutes; group 2 at 15 minutes; group 3 at 30 minutes; group 4 at 45 minutes; group 5 at 60; group 6 at 90 minutes; and group 7 at 120 minutes. At the time of sacrifice of a particular group, the brains of each mouse were quickly removed and dissected on ice. The following regions were collected weighed and assayed for radioactivity: cerebellum, hippocampus, striatum, parietal cortex, thalamus. The following values, averaged over each group of three mice, for the percentage of the administered dose of compound II/gram of brain tissue (%ID/g), were found in the following brain regions at five minutes post injection: striatum (6.19% injected dose/gram tissue); thalamus (4.76%); cortex (4.01%); cerebellum (3.76%); and hippocampus (3.41%). Striatum binding levels demonstrated highest specific binding defined as striatum-cerebellum at 30 minutes post injection (i.e., 4.33%). These results are depicted graphically in FIG. 2 and FIG. 3.

### EXAMPLE 5

### Dose Response Experiment

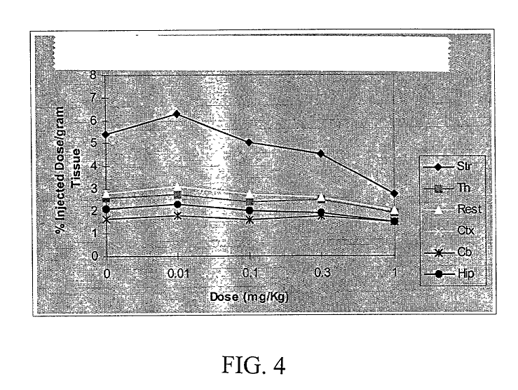

Male Charles River mice (CD-1) were divided into 5 groups of 3 mice each. Non-radiolabeled compound III was administered to each mouse in increasing doses as follows: group1, saline controls; group 2, 0.01 mg/kg; group 3, 0.1 mg/kg; group 4, 0.3 mg/kg; and group 5,1 mg/kg. Five minutes after the injection with compound III or the saline control, each mouse was administered compound II (421 μCi, 8 μg) by intravenous injection as above. Each mouse was sacrificed by cervical dislocation and brain tissue dissected and the radioactivity of each brain region assayed as described above. The values, averaged over each group of three mice, for the percentage of the administered dose of compound II/gram of brain tissue at each dosage of compound III for each brain region were calculated (FIG. 4 below). As shown in FIG. 4, binding in striatum was reduced by 6% at 0.1 mg/kg, 20% at 0.3 mg/kg and 52% at 1 mg/kg, respectively, relative to the saline control.

The present invention is not to be limited in scope by the specific embodiments disclosed in the Examples, which are intended as illustrations of a few aspects of the invention. Any embodiments that are functionally equivalent are within the scope of this invention. Indeed, various modifications of the invention, in addition to those shown and described herein, will become apparent to those skilled in the art and are intended to fall within the appended claims.

A number of references have been cited, the entire disclosures of which are incorporated herein by reference.

What is claimed is:

1. A method for detecting acetylcholinesterase in a brain of a patient, comprising:
(a) administering to the patient a detectable amount of a compound of a general formula I [chemical structure]
or a pharmaceutically acceptable salt thereof, the compound comprising one or more radioisotopic atoms selected from the group consisting of carbon-11, fluorine-18, iodine-123, and bromine-76, wherein:
Q is —(CH₂)ₘ—, —CH═CH—, —CHCH₃, —C(CH₃)₂, oxygen, sulfur, or —NR²;
X is oxygen or sulfur;
Y is —(CH₂)ₙ—;
L is phenyl or —(C₁-C₆)alkyl-phenyl, wherein said phenyl is optionally substituted with one or more —(C₁-C₆)alkyl or halo groups;
R¹ is —(C₁-C₆)alkyl;
R² is hydrogen or —(C₁-C₆)alkyl; and
n and m are independent integers ranging from 1 to 3;
with a proviso that the compound is not that of formula II [chemical structure]
(b) imaging the brain to generate a brain image showing a distribution and relative amounts of acetylcholinesterase in the brain.

2. The method of claim 1, wherein the compound is administered intravenously.

3. The method of claim 1, wherein the compound comprises a carbon-11 atom.

4. The method of claim 3, wherein R¹ comprises the carbon-11 atom.

5. The method of claim 1, wherein the imaging comprises performing PET or SPECT.

6. A method for diagnosing, estimating the severity of, or monitoring the progression of a dementia in a patient, comprising:
(a) administering to the patient a detectable amount of a compound of a general formula I [chemical structure]
or a pharmaceutically acceptable salt thereof, the compound comprising one or more radioisotopic atoms selected from the group consisting of carbon-11, fluorine-18, iodine-123, and bromine-76, wherein:
Q is —(CH₂)ₘ—, —CH═CH—, —CHCH₃, —C(CH₃)₂, oxygen, sulfur, or —NR²;
X is oxygen or sulfur;
Y is —(CH₂)ₙ—;
L is phenyl or —(C₁-C₆)alkyl-phenyl, wherein said phenyl is optionally substituted with one or more —(C₁-C₆)alkyl or halo groups;
R¹ is —(C₁-C₆)alkyl;
R² is hydrogen or —(C₁-C₆)alkyl; and
n and m are independent integers ranging from 1 to 3;
with a proviso that the compound is not that of formula II [chemical structure]
(b) imaging the brain of the patient to generate a brain image showing a distribution and relative amounts of acetylcholinesterase in the brain.

7. The method of claim 6, wherein the dementia is Alzheimer's disease.

8. The method of claim 6, wherein the compound is administered intravenously.

9. The method of claim 6, wherein the compound comprises a carbon-11 atom.

10. The method of claim 9, wherein R¹ comprises the carbon-11 atom.

11. The method of claim 6, wherein the imaging comprises performing PET or SPECT.

12. A method for detecting acetylcholinesterase in a brain of a patient, comprising:
(a) administering to the patient a detectable amount of a compound of a formula II 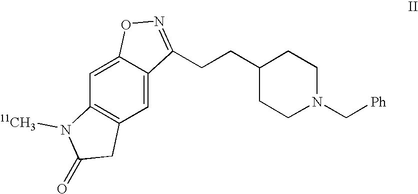
or a pharmaceutically acceptable salt thereof; and
(b) imaging the brain to generate a brain image showing a distribution and relative amounts of acetylcholinesterase in the brain.

13. The method of claim 12, wherein the compound is administered intravenously.

14. The method of claim 12, wherein the imaging comprises performing PET or SPECT.

15. A method for diagnosing, estimating the severity of, or monitoring the progression of a dementia in a patient, comprising:
(a) administering to the patient a detectable amount of a compound of a formula II [chemical structure]
